(19) United States
(12) Patent Application Publication
(10) Pub. No.: US 2026/0092927 A1
(43) Pub. Date: Apr. 2, 2026

(54) YEAST-LIQUID-HYBRID (YLH) HIGH-THROUGHPUT METHOD TO UNCOVER PROTEIN CONDENSATE CONSTITUENTS

(71) Applicant: The Trustees of Princeton University, Princeton, NJ (US)

(72) Inventors: José L. Avalos, Princeton, NJ (US); Daniel J. Cohen, Princeton, NJ (US); Lisset A. Duran, Princeton, NJ (US); Ka-Hei Siu, Princeton, NJ (US)

(73) Assignee: The Trustees of Princeton University, Princeton, NJ (US)

(21) Appl. No.: 19/342,978

(22) Filed: Sep. 29, 2025

Related U.S. Application Data
(60) Provisional application No. 63/699,921, filed on Sep. 27, 2024.

(51) Int. Cl. G01N 33/68; C12N 15/10; C12Q 1/20

(52) U.S. Cl. CPC G01N 33/6845; C12N 15/1037; C12Q 1/20

(57) ABSTRACT

The field of intracellular condensates is an emerging and rapidly growing field. Liquid membrane-less organelles composed of intrinsically disordered proteins (IDPs) are implicated in many critical functions of the cell, including signaling and metabolic pathways, transcription, translation, and the cell cycle. However, understanding of their basic biological and biochemical mechanisms is still limited by a lack of high-throughput methods to identify their protein constituents. The yeast-liquid-hybrid (YLH) method disclosed herein, inspired by the classical yeast-two-hybrid (Y2H) screen, can facilitate high-throughput identification of proteins that partition into protein condensates (e.g., liquid, gel-like, or solid condensates) in living cells. The development of this method will allow for the discovery of protein interactions at a much larger scale than currently possible, and the ability to screen large chemical collections to identify drug candidates to treat diseases associated with pathological protein condensates.

### CROSS-REFERENCE TO RELATED APPLICATIONS

The present application claims priority to U.S. Provisional Patent Application No. 63/999,921, filed Sep. 27, 2024, the contents of which are incorporated by reference herein in its entirety.

### TECHNICAL FIELD

Disclosed is a high-throughput technique for considering protein-protein interactions, and for uncovering protein condensate constituents.

### BACKGROUND

The field of intracellular condensates is an emerging and rapidly growing field. Liquid membrane-less organelles composed of intrinsically disordered proteins (IDPs) are implicated in many critical functions of the cell, including signaling and metabolic pathways, transcription, translation, and the cell cycle. However, understanding of their basic biological and biochemical mechanisms is still limited by a lack of high-throughput methods to identify their protein constituents.

### BRIEF SUMMARY

In various aspects, a method for identifying protein-protein interactions may be provided. The method may include forming a first modified yeast strain by introducing a Bait plasmid into a first yeast stain. The Bait plasmid may have a nucleic acid sequence that encodes a protein of interest (a Bait protein) fused to a Gal4 DNA Binding Domain (Gal4-DBD) under control of a first promoter. The Bait plasmid contains a naturally occurring Bait sequence, or may include a synthetic Bait sequence. The Bait protein may include an intrinsically disordered protein region (IDR), such as a FUS or a DDX4 IDR. The protein of interest may optionally contain a dimerization domain configured for direct protein binding. The first yeast strain may have an allele encoding mating-type-specific transcription factors, such as either a MATa or MATalpha allele.

Forming the modified yeast may then include introducing a Trans element (consisting of a nucleic acid sequence encoding a free Bait protein, the free Bait protein being free from fusion to Gal4-DBD), into the first yeast strain either through an episomal plasmid or by genomic integration. The Trans element may include an intrinsically disordered protein (IDP) or intrinsically disordered region (IDR). This may make a condensate around the Bait-Gal4-DBD protein bound to a promoter containing a DNA sequence to which the Gal4-DBD binds upstream of a selective marker.

The method may include introducing a Prey plasmid. In some embodiments, this may be done by introducing the Prey plasmid into a second yeast strain. In some embodiments, this may be done by introducing a library of Preys in the first strain. The Prey plasmid may include a Prey protein fused to a Gal4 Activation Domain (Gal4-AD) under a second promoter (which may be the same, or different, from the first promoter). The Prey plasmid may contain a Prey sequence derived from an organism that is the same, or different, from an organism from which the Bait sequence contained in the Bait plasmid is derived. The Prey plasmid may contain a synthetic Prey sequence. The Prey protein may optionally be free of a dimerization domain configured for direct protein binding to the Bait-Gal4-DBD protein. Preferably, the second yeast strain may have whichever of the MATa or MATalpha allele was not present in the first yeast strain. Preferably, a reporter gene may be genomically integrated into the second yeast strain.

The method may include, for each second modified yeast strain (if utilized), forming a mated yeast by mating the first modified yeast strain and the second modified yeast strain, then plating each mated yeast using a selective media in which the reporter genes are essential and incubating under incubation conditions configured to facilitate protein phase condensation. In some instances, a level of adenine in a growth medium for incubation may be configured to permit growth of strains containing the Trans element, the Bait protein, and Prey protein, but not strains containing only Trans element and the Bait protein. In certain aspects, the level of adenine may be 1%-5% by weight of the growth medium.

The method may then include allowing a putative Prey protein (e.g., one of the proteins expressed by the sequence in the Prey plasmid) to partition into a droplet or condensate formed by the Bait protein and Trans element, inducing expression of the essential gene, resulting in a viable colony.

The method may include identifying the putative Prey protein (e.g., that enabled colony growth under selection conditions) by sequencing the Prey plasmid containing a sequence encoding for a putative protein that partitions into the droplet or condensate. The method may include re-plating and re-incubating to eliminate one or more background colonies.

The method may include creating a Prey library by fusing at least one of a plurality of screening proteins to the Gal4-AD.

The method may include disrupting a droplet or condensate by changing a temperature above a temperature used for incubation and causing loss of the viable colony. The method may include inducing or inhibiting the formation of a condensate that brings Bait-Gal4-DBD, Free Bait and Prey-Gal4-AD by exposing the viable colony to a chemical agent.

In certain aspects, a method for drug screening against protein condensates may be provided. The method may include forming a first modified yeast strain by introducing a Bait plasmid into a first yeast stain and then introducing a Trans element into the first yeast strain by episomal plasmid or genomic integration to enable condensate formation at a first promoter to which Bait-Gal4-DBD binds. The Bait plasmid may have a nucleic acid sequence encoding a protein of interest fused to a Gal4 DNA Binding Domain (Gal4-DBD). The first strain may have either a MATa or MATalpha allele.

The method may include forming a second modified yeast strain by introducing a Prey plasmid into a second yeast strain, the Prey plasmid including a nucleic acid sequence for a Prey protein fused to a Gal4 Activation Domain (Gal4-AD) under a second promoter, the second yeast strain having the other of the MATa or MATalpha allele, where a reporter gene has been genomically integrated into the second yeast strain.

The method may include forming a mated yeast by mating the first modified yeast strain and the second modified yeast strain, and growing the mated yeast into a plurality of containers or locations (such as multi-well plates, a plurality of trays, etc.), each container or location having a selective media in which the reporter genes are essential. The method may include exposing at least some of the containers or locations to at least one small molecule from a library of small molecules, and incubating under incubation conditions configured to facilitate protein phase condensation.

The method may include identifying at least one first container or location in which cells do not grow or form colonies at the same rate or level as a strain not exposed to the small molecule(s), indicating that condensate formation did not occur after incubation or is atypical. The method may include identifying the small molecule associated with the first container(s) or location(s).

The method may include identifying at least one second container or location in which cells grow or form colonies at a higher rate or level as the control strain not exposed to the small molecule(s), indicating that condensate formation is enhanced by the small molecule after incubation. The method may include identifying the small molecule associated with the second container(s) or location(s).

### BRIEF DESCRIPTION OF FIGURES

FIG. 1 is a schematic illustration of a convention Yeast-Two-Hybrid (Y2H) technique.

FIG. 2 is a schematic illustration of an embodiment of a Yeast-Liquid-Hybrid (YLH) technique.

FIGS. 3A and 3B are flowcharts of different embodiments of methods.

FIG. 4 is a schematic representation of a Bait sequence.

FIG. 5 is a schematic representation of a Trans sequence.

FIG. 6 is a schematic representation of a Prey sequence.

FIG. 7 is a schematic representation of a Reporter sequence.

FIG. 8 is a flowchart of a method.

FIG. 9 are images of plates after incubation of a Bait/Prey system where different strength promoters and different selective media are used.

FIG. 10A is an image of a set of plates showing performance of a particular Bait/Prey system under different promoters and selective media.

FIG. 10B is an image of a set of plates showing that FUS alone cannot recruit RNA polymerase by itself without a Trans element.

FIGS. 10C and 10D are images showing fluorescent intensity (when a fluorescent tag was included in the system) under a CYC1p (10C) or GPDp (10D) promoter.

FIG. 11 is an image of a set of plates showing the impact of controlling the lelve of adenine in the selective media.

FIGS. 12 and 13 are images of a set of plates showing the impact of growth of different systems at an elevated temperature (12) and at room temperature (13).

FIG. 14 is a schematic representation of an embodiment of the disclosed Yeast-Liquid-Hybrid (YLH) method. In the absence of nuclear condensate formation, the co-expression of DB-FUS (DNA binding domain of Gal4p fused to full length FUS) and AD-FUSn (Activation Domain of Gal4p fused to FUSn) fails to reconstitute the Gal4 transcription factor. To overcome this limitation, a nuclear localization signal (NLS)-tagged FUS bait protein (“Free Bait”) is introduced into the nucleus to increase FUS IDR concentration and promote nuclear droplet formation, including around the DNA-bound Bait. Upon droplet formation, AD-FUSn partitions into the DB-FUS+Free-Bait condensate, enabling RNA polymerase recruitment and activation of the selection gene.

FIG. 15 is a set of images showing that yeast strain co-expressing AD-FUSn, BD-FUS, and Free Bait shows robust growth under YLH selection conditions (-HIS, -ADE), while the control strains without Free Bait (negative control, ‘NC’) or expressing Bait and Free Bait components containing mutant FUS with Y residues mutated to S failed to grow, suggesting the necessity for nuclear FUS condensation to drive transcriptional activation.

FIG. 16 is a graph showing quantification of droplet number per cell in the nucleus and cytosol for strains expressing high and low levels of Free-Bait. Data are presented as mean±SD; n=20 cells.

FIG. 17 shows maximum intensity projections of confocal microscopy images of YLH components expressed individually, highlighting nuclear droplet formation; the inset shows magnified view of a puncta-positive nucleus.

### DETAILED DESCRIPTION

Disclosed herein is a Yeast-Liquid-Hybrid (YLH) high-throughput method that can be used to, e.g., uncover protein condensate constituents. The hybrid is not made by the interaction of two or three proteins, but by the interaction of a protein in the condensate (e.g., a liquid, gel-like, or solid condensate) made by a Trans element with the DB-Bait.

The YLH method disclosed herein, inspired by the classical yeast-two-hybrid (Y2H) screen, can facilitate high-throughput identification of proteins that partition into protein condensates in living cells. The protein condensates may preferably be liquid condensates, but other condensates, such as gel-like or solid condensates, can also be formed and utilized with the same technique. The development of this method will allow for the discovery of protein interactions at a much larger scale than currently possible, and the ability to screen large chemical collections to identify drug candidates to treat diseases associated with pathological protein condensates.

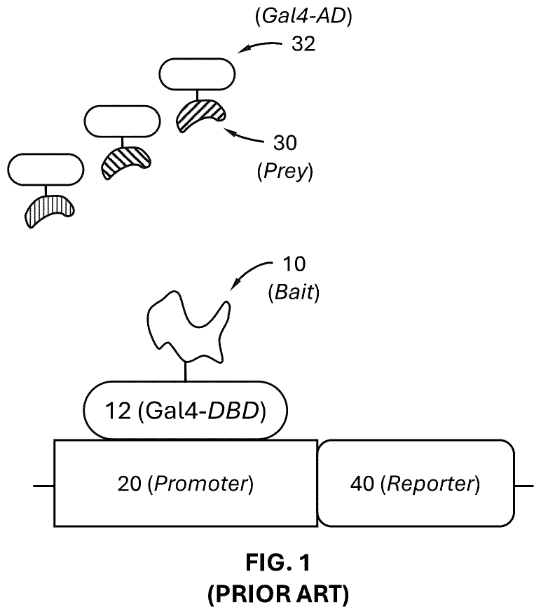

More particularly, the YLH process is derived from a modification to a classical high throughput screen method to identify protein-protein interactions using yeast called Yeast-Two-Hybrid (Y2H). Referring to FIG. 1, in the classical Y2H method, a protein of interest (the Bait) (10) is fused to the Gal4 DNA Binding Domain (Gal4-DBD) (12), which binds to the PGAL1 promoter (20). Separately, a library of proteins (the Preys) (30) to be screened for interactions with the Bait, are fused to the Gal4 Activation Domain (Gal4-AD) (32), which recruits RNA Polymerase to initiate transcription of a reporter gene (40) under the control of PGAL1. One can then screen for direct protein-protein interaction between the Bait and Prey contained in colonies that grow under conditions in which the reporter gene is essential for growth (for example PGAL1-HIS3, essential for growth in media lacking the essential nutrient histidine).

The classical YTH method is very effective at identifying protein interactions when both Bait and Pray are structured proteins that make specific protein-protein interactions. However, it is ineffective at identifying interactions between intrinsically disordered proteins (IDPs), which make weaker, less specific, and more transient interactions through protein condensates (including liquid, gel-like, or even solid condensates).

More particularly, the classical Y2H method cannot identify phase-separation dependent interactions. There is currently no robust high throughput method to systematically study the natural interactions of IDPs with protein condensates, nor to screen drug candidates to disrupt or induce the formation of protein condensates implicated in human disease, or the ability to design synthetic peptides that partition into synthetic condensates to make synthetic organelles.

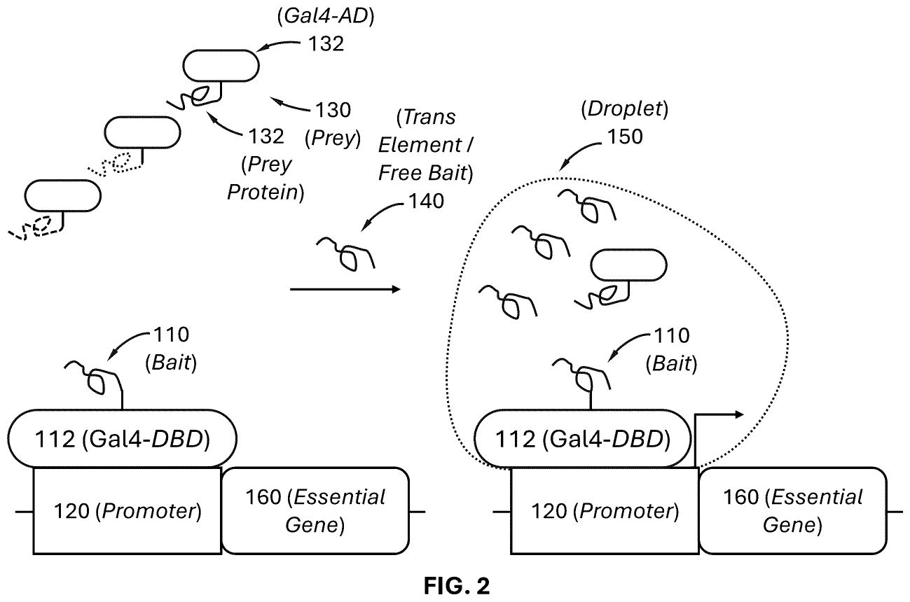

In contrast, the YLH method disclosed herein is effective at identifying interactions between intrinsically disordered Baits and Preys as they undergo a phase transition into protein condensates (for example, as liquid droplets). This is shown schematically in FIG. 2. In the disclosed YLH method, an IDP known to form phase separated droplets (Bait) (110) is genetically fused to Gal4-DBD (112) and a cDNA library derived from the same organism is used to generate a library of Preys (e.g., as shown in FIG. 2, each Prey (130) includes a Prey protein (130) fused to Gal4-AD (132)).

However, to identify the disordered Preys that interact with disordered Baits, it is important to introduce a new element in the assay, namely, a Trans element (140) consisting of the same IDP used to produce the Bait, but without it being fused to the Gal4-DBD (also called “Free Bait”). This Trans element (140) will then condense on the Bait (for example, making liquid droplets (150), such as nanodroplets), on to which the Prey can partition and induce expression of the essential gene (160). One typically expresses the Trans element at different levels to induce the appropriate amount of condensate (nanodroplets) to recruit the Prey.

Additionally, the screening protocol, media, and temperature may be optimized to allow for phase-separation mediated growth in media in which the gene reporter is essential. Disruption of these nanodroplets by chemical of physical means can also be screened with the disclosed YLH assay, as demonstrated by the temperature sensitivity of the assay to droplet formation and thus expression of the essential gene.

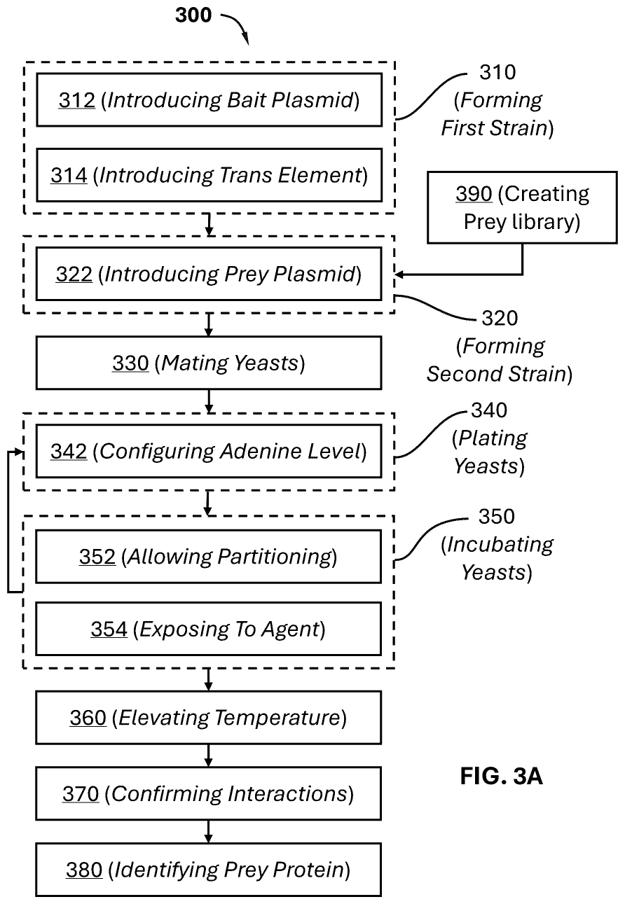

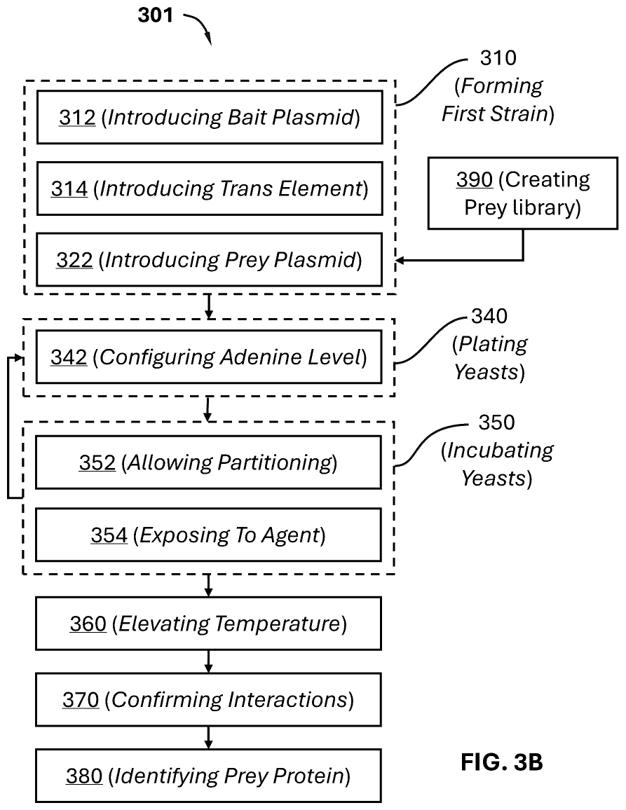

Referring to FIG. 3A, in various aspects, a method (300) for identifying protein-protein interactions may be provided. The method may include forming (310) a first modified yeast strain by introducing (312) a Bait plasmid into a first yeast stain. The first yeast strain may have either a MATa or MATalpha allele. As is known in the art, haploid yeast cells come in two mating types, “a” and “alpha”, each producing specific pheromones to identify and interact with the opposite type. In a Saccharomyces yeast strain, the sex type expression locus is a MAT locus which comprises two alleles: MATa and MATalpha.

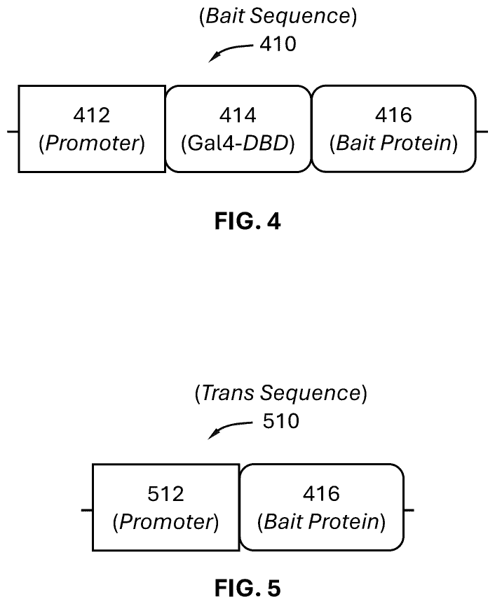

Referring to FIG. 4, the Bait plasmid may have a nucleic acid sequence (e.g., Bait sequence (410)) that encodes a protein of interest (a Bait protein) (416) fused to a Gal4 DNA Binding Domain (Gal4-DBD) (414) under control of a Bait promoter (412). The Bait sequence may be a naturally occurring sequence, or may be a synthetic sequence.

In preferred embodiments, the Bait protein may include any intrinsically disordered protein region (IDR), such as a FUS or a DDX4 IDR. The protein of interest may optionally contain a dimerization domain configured for direct protein binding with the Free Bait protein. As used herein. the term “dimerization domain” generally refers to a ligand-binding domain that binds to a binding moiety of a bi-specific ligand to form a dimer, or in some examples, enables spontaneous dimerization of two peptides. A dimerization domain can allow homotypic and/or heterotypic interactions. Dimerization domains can also be drug-dependent (i.e., depending on the presence of, e.g., a small molecule in order to function). Dimerization domains that enable spontaneous dimerization include but are not limited to leucine zipper, zinc finger domain, or cysteine knot domains.

As used herein, the term “small molecule” generally refers to a molecule that has a molecular weight of less than 5000 g/mol, less than about 2500 g/mol, less than about 2000 g/mol, less than about 1500 g/mol, less than about 1000 g/mol, less than about 800 g/mol, or less than about 500 g/mol.

Referring to FIG. 3A, forming (310) the first modified yeast strain may include introducing (314) a Trans element into the first yeast strain either through an episomal plasmid or by genomic integration.

Referring to FIG. 5, the Trans element generally consists of a a nucleic acid sequence (e.g., Trans sequence (510)) encoding a free Bait protein (416), which may under control of a Trans promotor (512). The Trans promoter may be the same or different from the Bait promoter (412). The free Bait protein is free from fusion to Gal4-DBD (e.g., it is preferably just the Bait protein, by itself).

The Trans element may include an intrinsically disordered protein (IDP). As used herein, the term “intrinsically disordered protein” generally refers to a protein that lacks a fixed or ordered three-dimensional structure. In certain instances, IDPs may adopt a fixed three-dimensional structure after binding to other macromolecules. Some IDPs may be characterized by a low portion of bulky hydrophobic amino acids and a high proportion of polar and charged amino acids (aka “low hydrophobicity” amino acids). This property leads to interactions with, e.g., water molecules. Examples of IDPs include, e.g., FUS, DDX4, p53, or Tau.

This Trans element enables condensate (which may be a nanocondensate, e.g., a condensate at the nanoscale, e.g., on the order of 1-100 nm in diameter) formation around the Bait-Gal4-DBD protein bound to a promoter containing a nucleic acid sequence (e.g., a DNA sequence) to which the Gal4-DBD binds. This can be seen in FIG. 2, where the element (140) induces the formation of the droplet (150), the droplet being formed around the Bait (110) fused to the Gal4-DBD (112)), which is bound to promotor (120).

Referring to FIG. 3A, the method may include forming (320) at least one second modified yeast strain. This may be done by, e.g., introducing (322) a Prey plasmid into a second yeast strain.

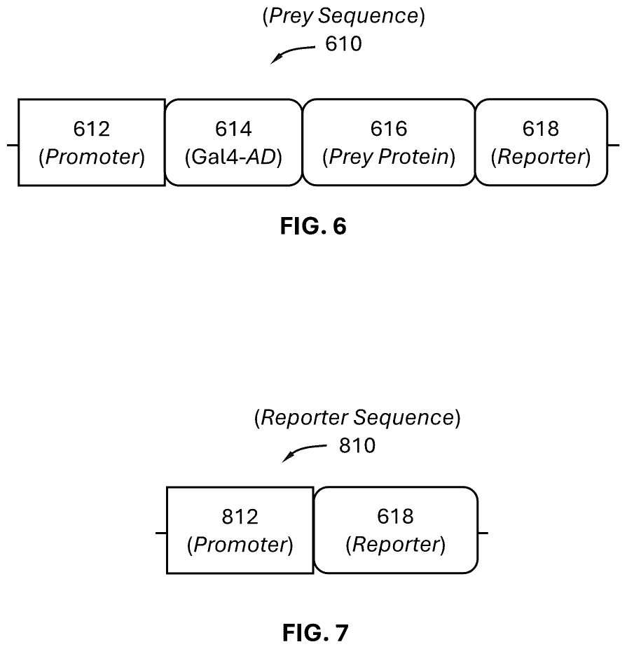

Referring to FIG. 6, the Prey plasmid may include a Prey sequence (610) encoding a Prey protein (616) fused to a Gal4 Activation Domain (Gal4-AD) (614) under a Prey promoter (612). The second promotor may be the same, or different, from the first promoter.

Any Prey protein is envisioned. For example, the Prey plasmid may contain a Prey sequence derived from an organism that is the same, or different, from an organism from which the Bait sequence contained in the Bait plasmid is derived. The Prey plasmid may contain a synthetic Prey sequence. The Prey protein may optionally be free of a dimerization domain configured for direct protein binding.

The second yeast strain may have whichever of the MATa or MATalpha allele was not present in the first yeast strain. As will be understood, this allows the first and second strains to eventually be mated.

A reporter gene may be genomically integrated into the second yeast strain. As seen in FIG. 7, a separate Reporter sequence (810) may be provided, having the reporter gene (814) under control of a Reporter promoter (812). The Reporter promoter may be the same or different from the Prey promoter (612). In a preferred embodiment, the Bait-Gal4-DBD may attach to the Reporter promoter (See, e.g., FIG. 2).

The method may include, for each second modified yeast strain, forming a mated yeast by mating (330) the first modified yeast strain and the second modified yeast strain. Each mated yeast may then be plated (340) using a selective media in which the reporter genes are essential. In some examples, an optimized selective media used a dropout synthetic complete supplement mixture (SC) of amino acids (e.g., SC-Tryp-Leu-His-Ade)+2% glucose, and optionally some amount of adenine, such as 1% adenine, in which the reporter genes (e.g., HIS3) are essential. In some instances, a level of adenine in a growth medium for incubation may be configured (342) to permit growth of strains containing the Trans element, the Bait protein, and Prey protein, but not strains containing only Trans element and the Bait protein. As will be understood, the exact level may depend on the particular strain, but in certain aspects, the level of adenine may be 1%-5% by weight of the growth medium.

Referring to FIG. 3B, it may be seen that, rather than forming a second yeast strain and mating, one or more Prey plasmids may be introduced into the first strain.

The method may include incubating (350) under incubation conditions configured to facilitate protein phase condensation. For example, plates may be incubated at temperature of 30° C. (as common to Y2H method) or lower temperatures (e.g., room temperature) to facilitate protein phase condensation.

The method may include allowing (352) a putative Prey protein (e.g., one of the proteins expressed by the sequence in the Prey plasmid) to partition into a droplet or condensate formed by the Bait protein and Trans element, inducing expression of the essential gene, resulting in a viable colony. The method may include inducing a droplet by exposing (354) a viable colony or a potentially viable colony to a chemical agent.

Any appropriate chemical agent may be introduced. As used herein, the term “chemical agent” encompasses any chemical molecule, or chemical element, or a combination of chemical molecules and/or chemical elements. For example, the term “chemical agent” encompasses proteins (e.g., having at least 50 covalently linked amino acid units) and peptides (e.g., having from 2 to 49 covalently linked amino acid units)

The method may include disrupting a droplet or condensate by changing (360) a temperature above a temperature acceptable for incubation and causing loss of a viable colony. For example, loss of colony formation at elevated temperatures (e.g., 30° C.-37° C.) is indicative of droplet disruption.

The method may include interactions between Prey and Bait may be confirmed (370), e.g., biochemically, genetically, or through imaging analysis, using known techniques. Such confirmation may be useful for reducing the risk of a false-positive.

The method may include identifying (380) the putative Prey protein (e.g., that formed the viable colony) by sequencing the Prey plasmid containing a sequence encoding for a putative protein that portions into the droplet or condensate. The method may include re-plating and re-incubating to eliminate one or more background colonies.

The method may include creating (390) a Prey library by fusing a least one of a plurality of screening proteins to the Gal4-AD, including generating the fusion gene. The fusion genes may be used as part of the Prey sequence.

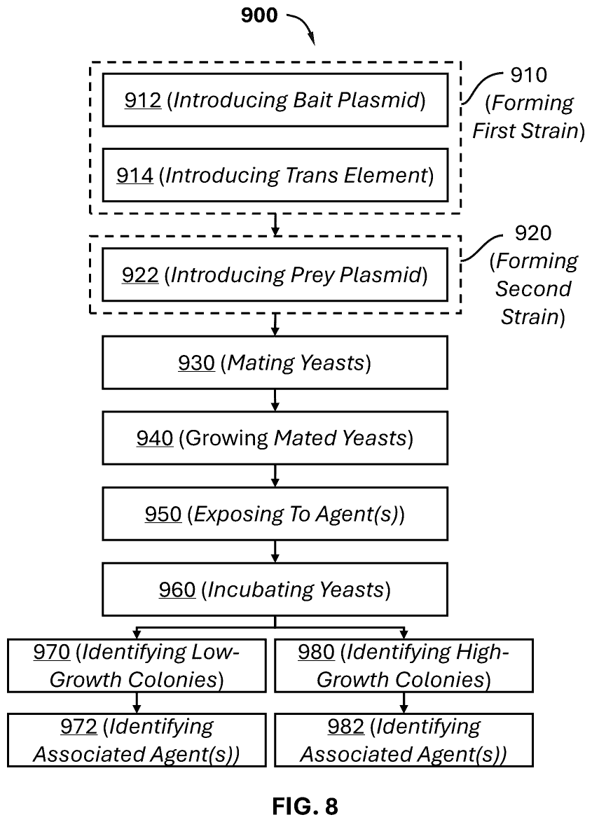

Similarly, a method for drug screening against protein condensates may be provided. Referring to FIG. 8, the method (900) may include forming (910) a first modified yeast strain by, e.g., introducing (912) a Bait plasmid into a first yeast stain and introducing (914) a Trans element into the first yeast strain by episomal plasmid or genomic integration to enable condensate formation at a first promoter to which Bait-Gal4-DBD binds. As disclosed herein, the Bait plasmid may include a nucleic acid sequence (e.g., Bait Sequence (410)) encoding a protein of interest (e.g., Bait Protein (416)) fused to a Gal4 DNA Binding Domain (Gal4-DBD) (414), which may be under the control of a Bait promoter (412). As disclosed herein, a Trans plasmid may include a nucleic acid sequence (e.g., Trans sequence (510)) encoding a free Bait protein (514) under the control of a Trans promoter (512). The first strain may have either a MATa or MATalpha allele.

The method may include forming (920) a second modified yeast strain by introducing (922) a Prey plasmid into a second yeast strain. As disclosed herein, the Prey plasmid may include a nucleic acid sequence (e.g., Prey Sequence (610)) for a Prey protein (616) fused to a Gal4 Activation Domain (Gal4-AD) (614) under control of a Prey promoter (612). The second yeast strain should have the other of the MATa or MATalpha allele (to allow the first and second yeast strains to mate). A reporter gene (618) may have been genomically integrated into the second yeast strain as disclosed herein.

The method may include forming a mated yeast by mating (930) the first modified yeast strain and the second modified yeast strain, and growing (940) the mated yeast into a plurality of containers or locations (such as multi-well plates, a plurality of trays, etc.), each container or location having a selective media in which the reporter genes are essential. The method may include exposing (950) at least one container or location to one or more chemical or biological agent(s), such as one or more small molecules from a library of small molecules, and incubating (960) under incubation conditions configured to facilitate protein phase condensation.

The method may include identifying (970) at least one first container or location in which cells do not grow or form colonies at the same rate or level as a control (e.g., a strain not exposed to the one or more agent(s)) (that is, identifying a “low-growth” colony), indicating that condensate formation did not occur after incubation or is atypical. The method may include identifying the agent(s) associated with the first container(s) or location(s).

The method may include identifying (980) at least one second container or location in which cells grow or form colonies at a higher rate or level as the control (that is, identifying a “high-growth” colony), indicating that condensate formation is enhanced by the agent(s) after incubation. The method may include identifying the agent(s) associated with the second container(s) or location(s).

As will be understood, the disclosed YLH may be used as a high throughput assay to identify new proteins that interact with protein condensates of interest, starting with those associated with human health or disease, e.g., by selecting an appropriate Bait protein.

The disclosed YLH may be used for drug screens against protein condensates. Having a strain whose growth depends on the formation of a specific protein condensate of interest could be used to screen large collections of small molecules to identify those that can inhibit condensate formation and thus strain growth. This is a potentially revolutionary method to identify drugs that target all kinds of protein condensates.

The disclosed YLH may be used for screening, to identify drugs that induce protein condensate formation. If a disease is caused by the lack of a normal protein condensate formation (due to mutation of IDRs or physiological changes that repress condensate formation), the YLH method could be used to screen for drug candidates that restore formation of the protein condensate and restore health to the cell.

The YLH may also be used to study and develop engineered disordered peptides that can recruit specific proteins into synthetic condensates. One can envision screening synthetic peptides against Y2H libraries and identifying novel peptides that can sequester and/or inhibit important targets. This could find multiple applications in the development of synthetic organelles for various biotechnological applications.

### Example 1: A Traditional Y2H Can Identify FUS Self-Interaction but not Phase Separation

The traditional Y2H is a powerful tool that allows for the identification of direct protein-protein interactions. To test that the Y2H techniques are not capable of identifying phase separation, full length Fus (FUS) were fused to the Gal4-DBD to create Bait-Fus. Separately, either full FUS or the FUS N-terminal disorder region (FUSIDR) was fused to the Gal4-AD to create Prey-Fus and Prey-FUSIDR. Full FUS contains a dimerization domain capable of direct protein binding, which has been deleted in the FUSIDR stretch. When grown in Y2H selective conditions (SC-Tryp-Leu-His-Ade at 30° C.), one sees robust growth for Prey-FUS but not for Prey-FUSIDR containing strains. From these results, one concludes that a traditional Y2H cannot identify phase dependent protein interaction.

### Example 2: Development of the Yeast Liquid Hybrid System

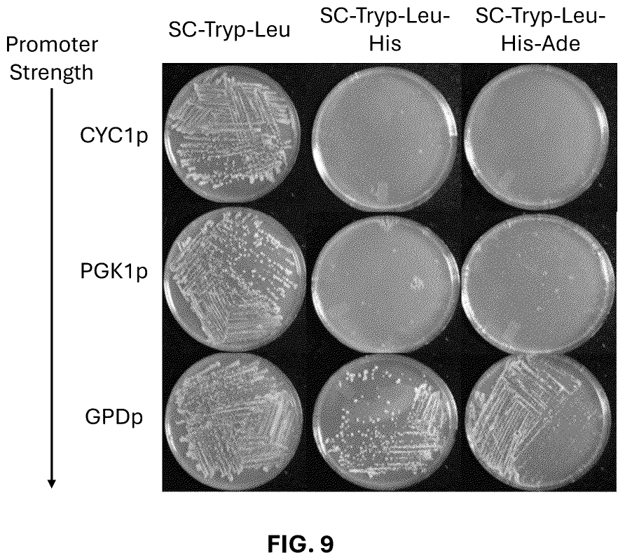

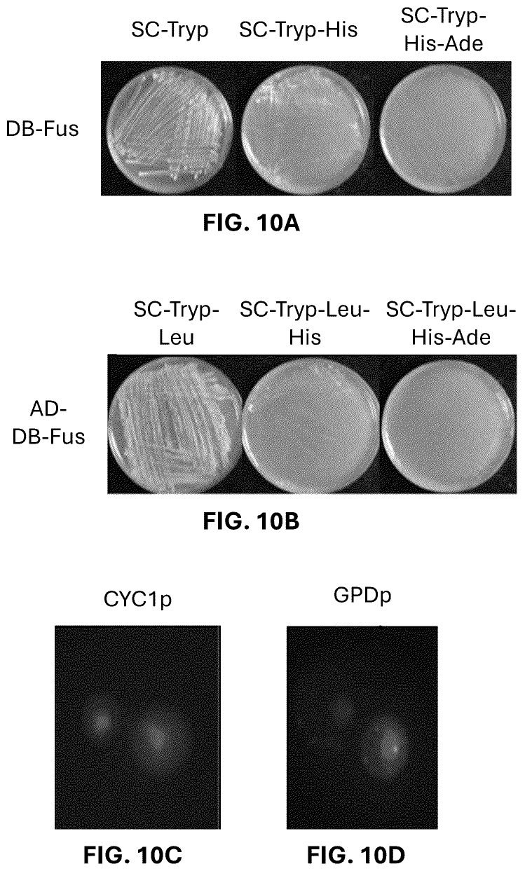

Since a traditional Y2H cannot identify phase separation, two independent but complementary approaches were developed to identify novel protein partners that interact through protein phase separations. The first relies on expression in trans of a nuclearly localized free FUS (not fused to either Gal4-DBD, or Gal4-AD), or what was called herein the Trans-FUS element. One sees that strong expression of Trans-FUS under a TDH3 promoter recapitulates the ability of the Bait-FUS and Prey-FUSIDR combination to grow under selective conditions. FIG. 9 shows a same Bait-FUS and Prey-FUSIDR combination with different combinations of promoters and selective media. FIGS. 10A and 10B show the results of tests to confirm whether FUS can recruit RNA polymerase by itself when expressed under GPD. In FIG. 10A, “DB-Fus” refers to a DNA binding domain of Gal4p fused to full length FUS. In FIG. 10B, “AD-DB-Fus” refers to two separate constructs: AD-Empty (Activation Domain of Gal4p not bound to anything) and DB-Fus. The dependence on Trans-element expression was confirmed by independent experiments with Tardigrade Intrinsically Disordered proteins. FIGS. 10C and 10D are images further indicating how decreasing the strength of the promoter impacts performance.

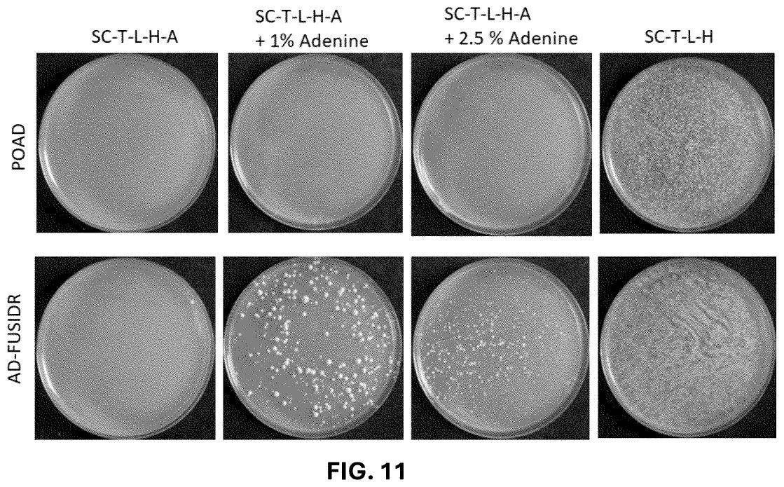

In addition, the media used for the mated diploid strain YLH growth assay was optimized. This was achieved by carefully titering the levels of adenine to an optimal value of 1-5% adenine (see FIG. 11), which permits growth of strains containing the Trans-element and both Bait-FUS and Prey-FUSIDR (“AD-FUSIDR”) but not strains that only contain the Trans-element and Bait-FUS (and no Prey) (Gal4-AD without being fused to any protein) (“POAD”).

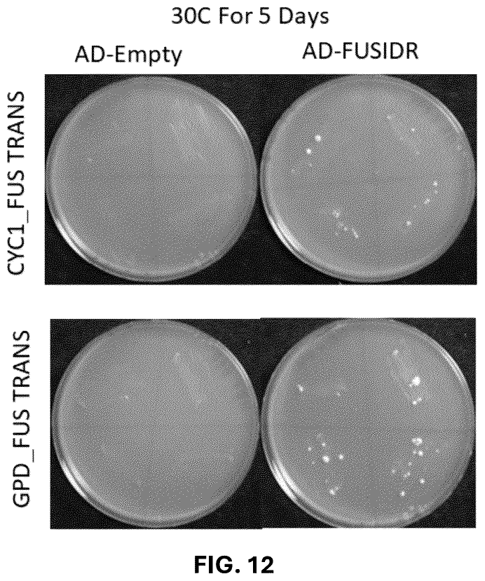

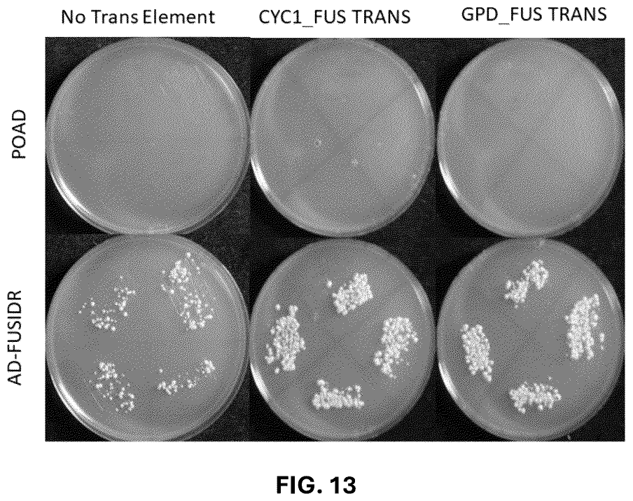

Finally, as seen in FIGS. 12 and 13, it was shown that raising the temperature from room temperature to 30° C. can inhibit growth of strains containing both Bait-FUS, Prey-FUSIDR (“AD-FUSIDR”), and Trans-FUS due to disruption of the protein condensate that enables transcription of the essential gene markers. This is a proof of principle that the assay could be used to screen for physical or chemical conditions that disrupt protein condensate formation. In FIG. 12, “AD-Empty”refers to an activation domain of Gal4p not fused to any protein.

### Example 3—Confirming Interaction are Mediated by Phase Separation

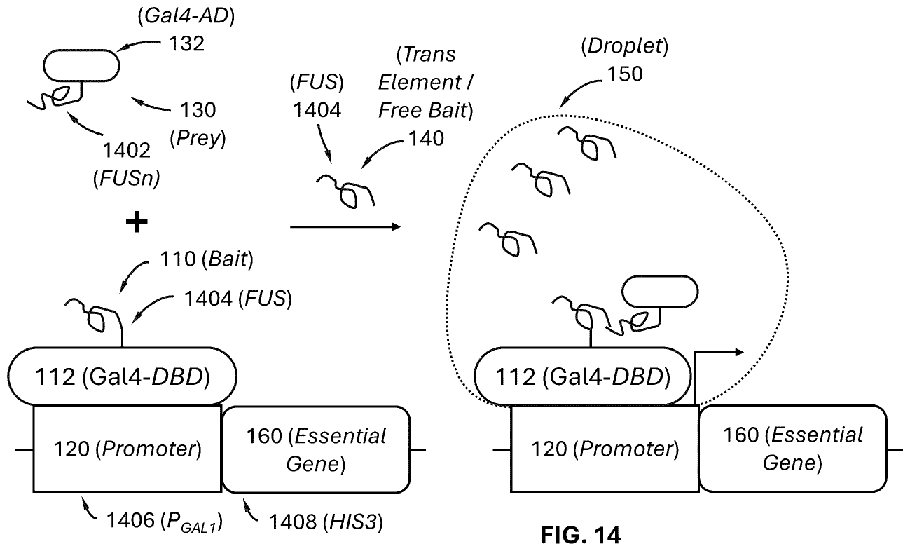

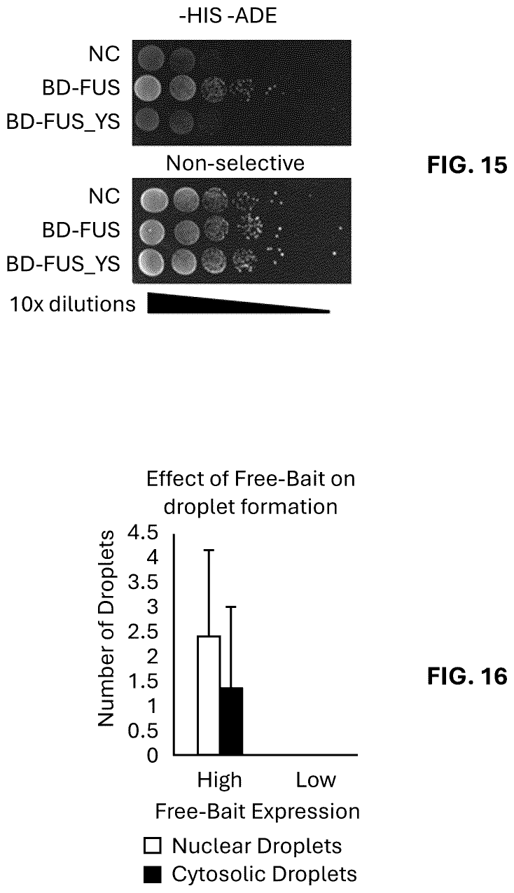

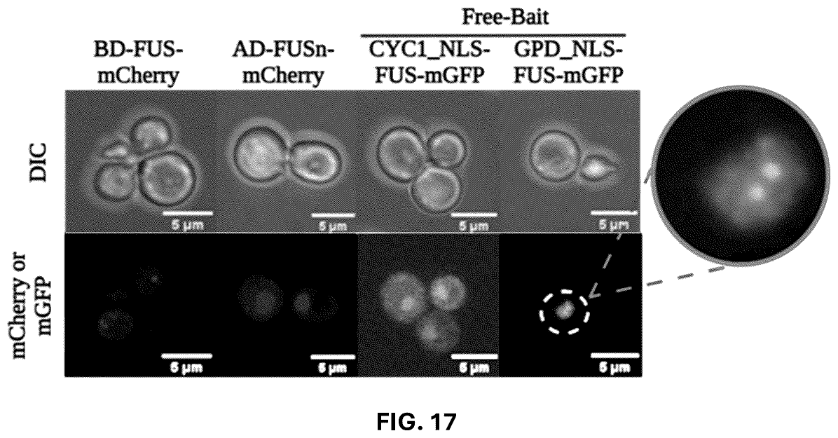

To validate the ability of YLH to detect phase-separation-dependent interactions, it was tested whether the disclosed techniques could detect FUS LLPS-dependent interactions. Starting with the strain containing FUS as Bait and Gal4-AD fused only to FUSn, which failed to grow in selective media, FUS was introduced as Free Bait. See FIG. 14. Having this third component enabled the cell to grow on selective media. See FIG. 15. This growth, however, was abolished when using mutant FUS in the Bait (BF-FUS) and Free Bait containing Y->S mutations, which eliminate the π-π interactions driving LLPS of FUS. See FIGS. 15-17. These results show that YLH can genetically detect condensate formation and its potential use as a high-throughput method for detecting LLPS-dependent interactions that overcomes key limitations of current screening methods. This data confirms that the interactions are mediated by phase separation, which depend on the π-π interactions that are abolished by the Y→S mutations.

Therefore, the disclosed YLH method could potentially be used for screening small molecules that inhibit, enhance, or modify protein condensates in search of potential drug candidates that target protein condensates associated with human health and disease.

What is claimed:

1. A method for identifying protein-protein interactions, comprising:
forming a first modified yeast strain by (i) introducing a Bait plasmid into a first yeast stain, the Bait plasmid having a nucleic acid sequence encoding a protein of interest (Bait protein) fused to a Gal4 DNA Binding Domain (Gal4-DBD) (a Bait-Gal4-DBD protein) under control of a first promoter, the first yeast strain having either a MATa or MATalpha allele, and then (ii) introducing a nucleic acid sequence encoding a Trans element consisting of a free Bait protein, the free Bait protein being free from fusion to Gal4-DBD, into the first modified yeast strain either through an episomal plasmid or by genomic integration, which makes a condensate around the Bait-Gal4-DBD protein bound to a target promoter containing a DNA sequence to which the Gal4-DBD binds upstream of a selective marker;
forming at least one second modified yeast strain by introducing a Prey plasmid into a second yeast strain, the Prey plasmid including a nucleic acid sequence encoding a Prey protein fused to a Gal4 Activation Domain (Gal4-AD) under control of a second promoter, the Prey protein optionally being free of a dimerization domain configured for direct protein binding to the Bait-Gal4-DBD protein, the second yeast strain having the other of the MATa or MATalpha allele not present in the first modified yeast strain, where a reporter gene has been genomically integrated into the second modified yeast strain;
for each second modified yeast strain, forming a mated yeast by mating the first modified yeast strain and the second modified yeast strain;
plating each mated yeast using a selective media in which the reporter genes are essential and incubating under incubation conditions configured to facilitate protein phase condensation; and
allowing a putative Prey protein to partition into a droplet or condensate formed by the Bait protein and Trans element, inducing expression of the essential gene, resulting in a viable colony.

2. The method of claim 1, wherein the protein of interest contains a dimerization domain configured for direct protein binding with the Free Bait protein,

3. The method according to claim 1, further comprising identifying the Prey protein that enabled colony growth under selection conditions by sequencing the Prey plasmid containing a sequence encoding for a putative protein that partitions into the droplet or condensate.

4. The method according to claim 1, further comprising re-plating and re-incubating to eliminate one or more background colonies.

5. The method according to claim 1, further comprising creating a Prey library by fusing at least one of a plurality of screening proteins to the Gal4-AD.

6. The method according to claim 1, wherein the Prey plasmid contains a Prey sequence derived from an organism that is different from an organism from which the Bait sequence contained in the Bait plasmid is derived.

7. The method according to claim 1, wherein the Prey plasmid contains a Prey sequence derived from an organism that is the same as an organism from which the Bait sequence contained in the Bait plasmid is derived.

8. The method according to claim 1, wherein the Prey plasmid contains a synthetic Prey sequence.

9. The method according to claim 1, wherein the Bait plasmid contains a synthetic Bait sequence.

10. The method according to claim 1, further comprising disrupting a droplet or condensate by changing a temperature from the temperature used for incubation and causing loss of the viable colony.

11. The method according to claim 1, further comprising inducing or inhibiting formation of a condensate that brings Bait-Gal4-DBD, Free Bait and Prey-Gal4-AD by exposing the viable colony to a chemical agent.

12. The method according to claim 1, wherein the Trans element includes an intrinsically disordered protein (IDP) or region (IDR).

13. The method according to claim 1, wherein the Bait protein includes an intrinsically disordered protein region (IDR).

14. The method according to claim 13, wherein the IDR is a FUS or a DDX4 IDR.

15. The method according to claim 1, wherein a level of adenine in a growth medium for incubation is configured to permit growth of strains containing the Trans element, the Bait protein, and Prey protein, but not strains containing only Trans element and the Bait protein.

16. The method according to claim 15, wherein the level of adenine is 1%-5% by weight of the growth medium.

17. A method for identifying protein-protein interactions, comprising:
forming a first modified yeast strain by:
introducing a Bait plasmid into a first yeast stain, the Bait plasmid having a nucleic acid sequence encoding a protein of interest (Bait protein) fused to a Gal4 DNA Binding Domain (Gal4-DBD) under control of a first promoter, the first yeast strain having either a MATa or MATalpha allele;
introducing a nucleic acid sequence encoding a Trans element consisting of a free Bait protein, the free Bait protein being free from fusion to Gal4-DBD, into the first yeast strain either through an episomal plasmid or by genomic integration, which makes a condensate around the Bait-Gal4-DBD protein bound to a target promoter containing a DNA sequence to which the Gal4-DBD binds upstream of a selective marker; and
introducing a library of Prey plasmids, each Prey plasmid including a nucleic acid sequence encoding a Prey protein fused to a Gal4 Activation Domain (Gal4-AD) under control of a promoter, the Prey protein optionally being free of a dimerization domain configured for direct protein binding to the Bait-Gal4-DBD protein;
plating each the first modified yeast strain using a selective media in which the reporter genes are essential and incubating under incubation conditions configured to facilitate protein phase condensation; and
allowing a putative Prey protein to partition into a droplet or condensate formed by the Bait protein and Trans element, inducing expression of the essential gene, resulting in a viable colony.

18. A method for drug screening against protein condensates, comprising:
forming a first modified yeast strain by (i) introducing a Bait plasmid into a first yeast stain and then (ii) introducing a Trans element into the first modified yeast strain by episomal plasmid or genomic integration to enable condensate formation at a first promoter to which a Bait-Gal4-DBD protein binds, the Bait plasmid having a nucleic acid sequence encoding a protein of interest fused to a Gal4 DNA Binding Domain (Gal4-DBD) (a Bait-Gal4-DBD protein), the first strain having either a MATa or MATalpha allele;
forming a second modified yeast strain by introducing a Prey plasmid into a second yeast strain, the Prey plasmid including the nucleic acid sequence encoding a Prey protein fused to a Gal4 Activation Domain (Gal4-AD) under control of a second promoter that partitions into a condensate formed between the Free Bait and the Bait-Gal4-DBD proteins, the second yeast strain having the other of the MATa or MATalpha allele, where a reporter gene has been genomically integrated into the second modified yeast strain;
forming a mated yeast by mating the first modified yeast strain and the second modified yeast strain;
growing the mated yeast into a plurality of multi-well plates or a plurality of trays, each well or tray having a selective media in which the reporter genes are essential, exposing at least one well or tray to a chemical or biological agent, and incubating under incubation conditions configured to facilitate protein phase condensation;
identifying at least one first well or tray in which cells do not grow or form colonies at the same rate or level as a strain not exposed to the chemical or biological agent, indicating that condensate formation did not occur after incubation or is atypical;
identifying the chemical or biological agent associated with the at least one first well or tray;
identifying at least one second well or tray in which cells grow or form colonies at a higher rate or level as the strain not exposed to the chemical or biological agent, indicating that condensate formation is enhanced by the chemical or biological agent after incubation; and
identifying the chemical or biological agent associated with the at least one second well or tray.
